(19) United States
(12) Patent Application Publication
(10) Pub. No.: US 2005/0192755 A1
(43) Pub. Date: Sep. 1, 2005

(54) Methods and systems for identification of macromolecules

(76) Inventors: Srinivasa Nagalla, Hillsboro, OR (US); Brian Searle, Portland, OR (US); Surendra Dasari, Portland, OR (US); Mark Turner, Portland, OR (US)

Correspondence Address: HELLER EHRMAN LLP, 275 MIDDLEFIELD ROAD, MENLO PARK, CA, 94025-3506, US

(21) Appl. No.: 10/789,424

(22) Filed: Feb. 27, 2004

(51) Int. Cl. G01N 33/53; G06F 19/00; G01N 33/48; G01N 33/50

(52) U.S. Cl. 702/19; 436/86

(57) ABSTRACT

A method is provided for identifying sequences of molecules and sequence modifications from mass spectrometry data. At least one de novo sequence is produced from mass spectrometry data of sequences of molecules,. At least one mass-based alignment is calculated between each de novo sequence and sequences in a sequence database. The molecular masses of molecules in the de novo sequence are compared to molecular masses of molecules in each sequence in the sequence database. Mass differences of modification sites are interpreted between the sequence in the sequence database and the de novo sequence that have been identified by the mass-based alignment as modifications identified in a modification catalog. At least one match score for the mass-based alignment is calculated that provides an indication of matching between the sequence in the sequence database and the de novo sequence. Sequences in the sequence database are identified from mass-based alignments in response to the match scores. Identifications of sequences in the sequence database are grouped from at least one de novo sequence into an identified macromolecule list that agrees with the de novo sequencing results.

### BACKGROUND OF THE INVENTION

1. Field of the Invention

This invention relates generally to methods and systems for the identification of macromolecules, and more particularly to methods and systems for the identification of proteins that match de novo sequences to homologous proteins.

2. General Statement Regarding References

The references cited in the present application are fully incorporated by reference, as though fully disclosed herein.

3. Description of the Related Art

Tandem mass spectrometry (MS/MS) is a commonly used tool in the high-throughput identification of proteins (Aebersold, R.; Mann, M. Nature 2003, 422, 198-207). Several software packages (Eng, J. K.; McCormack, A. L.; Yates, J. R. III J. Am. Soc. Mass Spectrom. 1994, 5, 976-989; Perkins, D. N.; Pappin, D. J. C.; Creasy, D. M.; Cottrel, J. S. Electrophoresis 1999, 20, 3551-3567; Field, H. I.; Fenyo, D.; Beavis, R. C. Proteomics 2002, 36-47; Denny, R.; Neeson, K.; Rennie, C.; Richardson, K.; Leicester, S.; Swainston, N.; Worroll, J.; Young, P. “The Use of Search Workflows in Peptide Assignment From MS/MS Data”, Association of Biomolecular Resource Facilities, ABRF '02: Biomolecular Technologies: Tools for Discovery in Proteomics and Genomics, Austin, Texas, Mar. 9-12, 2002) have been developed to identify proteins present in samples by utilizing the amino acid sequence specific information in MS/MS spectra of peptides to search protein sequence databases. These programs typically rely on a whole peptide mass filter, where candidate peptides from the database are compared to the unknown MS/MS spectra only if they match the experimental mass of the parent ion. This method is sufficiently reliable for high-throughput identification of proteins with known amino acid sequences. However, if the sample peptide differs from the database sequence due to sequence variation or database sequence errors, or if the peptide contains sites of post-translational modifications, the calculated mass from the database sequence may no longer match the measured mass.

In these cases, other strategies can be tried. One possibility is to create a database of proteins that contains all possible combinations of common modifications and to search unknown spectra against the new database (Yates, J. R. III; Eng, J. K.; McCormack, A. L.; Schieltz, D. Anal. Chem. 1995, 67, 1426-1436). However, with an exhaustive search, the number of combinations of modifications that must be tested can grow prohibitively large. Since it is more likely to have modified peptides of proteins already present in a sample, an efficient technique is to search for modified forms of only those proteins identified in an initial database search (Gatlin, C. L.; Eng, J. K.; Cross, S. T.; Detter, J. C.; Yates, J. R. III Anal. Chem. 2000, 72, 757-763; Pevzner, P. A.; Mulyukov, Z.; Dancik, V.; Tang, C. L. Genome Research 2001, 11, 290-299; Creasy, D. M; Cottrell, J. S. Proteomics 2002, 2, 1426-1434. This optimization method is used by AutoMod, a subroutine of ProteinLynx (Denny, R.; Neeson, K.; Rennie, C.; Richardson, K.; Leicester, S.; Swainston, N.; Worroll, J.; Young, P. “The Use of Search Workflows in Peptide Assignment From MS/MS Data”, Association of Biomolecular Resource Facilities, ABRF '02: Biomolecular Technologies: Tools for Discovery in Proteomics and Genomics, Austin, Texas, Mar. 9-12, 2002), and it can significantly reduce the search space. However, it does require the identification of at least one unmodified peptide in the initial database search, and is limited to identifying only peptides modified in ways represented by the new protein database.

Another technique is either to match ion series in MS/MS spectra to peptide sequences without using a stringent parent ion mass filter (Pevzner, P. A.; Mulyukov, Z.; Dancik, V.; Tang, C. L. Genome Research 2001, 11, 290-299; Clauser, K. R.; Baker, P.; Burlingame, A. L. “Peptide Fragment-Ion Tags from MALDI/PSD for Error-tolerant Searching of Genomic Databases”, Proceedings of the 44th ASMS Conference on Mass Spectrometry and Allied Topics, Portland, Oreg., May 12-16, 1996), or to match short peptide sequence motifs to features in spectra (Liebler, D. C.; Hansen, B. T.; Davey, S. W.; Tiscareno, L.; Mason, D. E. Anal. Chem. 2002, 74, 203-210). Using these methods, unanticipated protein modifications and sequence variations can be identified, provided that they do not alter the masses of a significant number of sequence-specific ions. However, both approaches often assign high scores to incorrect peptide identifications by chance, thereby limiting their application in high-throughput environments. As with AutoMod, the search space can be limited by identifying candidate proteins from unmodified peptides with database-searching programs; but again, extensive manual verification is often still required.

A third potentially high-throughput approach is GutenTag (Tabb, D. L.; Saraf, A.; Yates, J. R. III Anal. Chem. 2003, 75, 6415-6421), an automated and enhanced version of the sequence tag method (Mann, M.; Wilm, M. Anal. Chem. 1994, 66, 4390-4399; Pappin, D. J. C.; Rahman, D.; Hansen, H. F.; Bartlet-Jones, M.; Jeffery, W.; Bleasby, A. J. Mass Spectrom Biol. Sci. 1996, 135-150) that relies on searching for short amino acid sequences derived from tandem mass spectra in protein sequence databases. The GutenTag scoring system, which is a combination of five factors (a tag match, a mass-match on either side of the tag, and a tryptic-termini match on either side of the peptide), has been shown to be extremely reliable when identifying unmodified peptides. Unfortunately, the sequence tag method can still assign high scores to incorrect matches when attempting to identify modified peptides because only three of the five scoring factors can normally be used.

The manual interpretation of spectra, called de novo sequencing, is an approach that can sequence peptides without using database-searching programs (Johnson, R. S. “How to sequence tryptic peptides using low energy CID data”, http://www.abrf.org/ResearchGroups/

MassSpectrometry/EPosters/ms97quiz/Sequencing Tutorial.html). MS/MS spectra commonly contain short series of fragment ions where the mass differences between these ions match the masses of amino acids in the original peptide. These mass differences can be linked together to form partial or complete peptide sequences (McCormack, A. L.; Eng, J. K.; Yates, J. R. III Methods Companion Methods Enzymol. 1994, 6, 284-303). Areas of MS/MS spectra that cannot be assigned to standard amino acids may be due to incomplete peptide fragmentation, or to post-translational modifications that change the mass of amino acids. The manual interpretation of spectra is time consuming and requires considerable expertise. Fortunately, there are several commercial (Ma, B.; Zhang, K.; Hendrie, C.; Liang, C.; Li, M.; Doherty-Kirby, A.; Lajoie, G. Rapid Commun. Mass Spectrom. 2003, 17, 2337-2342; Scigelova, M.; Maroto, F.; Dufresne, C; Vazquez, J. “High-Throughput De Novo Sequencing”, 14th Meeting Methods of Protein Structure Analysis, Valencia, Spain, Sep. 8-12, 2002; Langridge, J. I.; Millar, A.; Young, P.; O'Malley, R.; Swainston, N.; Skilling, J.; Hoyes, J.; Richardson, K. “A Fully Automated Hierarchical Software Strategy for De Novo Sequencing of Whole Q-Tof Electrospray LC-MS/MS Datasets”, Proceedings of the 50th ASMS Conference on Mass Spectrometry and Allied Topics, Orlando, Fla., Jun. 2-6, 2002) and freely available (Fernandez-de-Cossio, J.; Gonzalez, J.; Betancourt, L.; Besada, V.; Padron, G.; Shimonishi, Y.; Takao, T. Rapid Commun. Mass Spectrom. 1998, 12, 1867-1878; Taylor, J. A.; Johnson, R. S. Anal. Chem. 2001, 73, 2594-2604; Uttenweiler-Joseph, S.; Neubauer, G.; Christoforidis, S.; Zerial, M.; Wilm, M. Proteomics 2001, 1, 668-682; Lu, B.; Chen, T. J. Comp. Biol. 2003, 10, 1-12) software packages that perform automated de novo sequencing. These programs take into consideration much of the possible variation in peptide fragmentation, and introduce the possibility of high-throughput, objective MS/MS sequencing.

One difficulty is that de novo sequencing algorithms often report several equally well-scoring sequences for a single spectrum, as well as ambiguous regions where the order or identity of two or more amino acids in the proposed sequence is uncertain. De novo sequencing algorithms also commonly misjudge the order of two or more residues, or mislabel residues as isobar equivalents. High mass accuracy can help alleviate the difficulty of assigning isobaric amino acids correctly. However, isomers such as leucine and isoleucine cannot be differentiated via low energy tandem mass spectrometry. Error-tolerant search engines must be used to differentiate sections of the de novo sequence that are inappropriately assigned by the sequencing algorithm from actual amino acid variations and post-translational modifications.

In the past, existing sequence alignment algorithms (Altschul, S. F.; Madden, T. L.; Schaffer, A. A.; Zhang, J.; Zhang,.Z.; Miller, W.; Lipman, D. J. Nucleic Acids Res. 1997, 25, 3389-3402; Pearson, W. R.; Lipman, D. J. Proc. Natl. Acad. Sci. USA 1988, 85, 2444-2448) have been modified in order to match de novo sequences to protein sequence databases. For example, MS-BLAST (Shevchenko, A.; Sunyaev, S.; Loboda, A.; Shevchenko, A.; Bork, P.; Ens, W.; Standing, K. G. Anal. Chem. 2001, 73, 1917-1926), MS-Shotgun (Huang, L.; Jacob, R. J.; Pegg, S. C.; Baldwin, M. A.; Wang, C. C.; Burlingame, A. L.; Babbitt, P. C. J. Biol. Chem. 2001, 276, 28327-28339), and FASTS (Mackey, A. J.; Haystead, T. A. J.; Pearson, W. R. Mol. Cell. Proteomics 2002, 1 139-147) can be used to align de novo sequences to database homologues using highly efficient sequence alignment algorithms. These programs use a modified mutation matrix to account for single residue isobars and can identify sequence differences or possible modification sites. It is possible to account for ambiguous regions by submitting a new search for every possible combination of amino acids that could add up to the summed mass of amino acids in that region. As the number of ambiguous regions in a de novo sequence grows, it quickly becomes more difficult to interpret the search results. Another program, CIDentify (Taylor, J. A.; Johnson, R. S. Rapid Commun. Mass Spectrom. 1997, 11, 1067-1075), attempts to correct for de novo sequencing errors by employing a re-scoring approach. After an alignment is made, unresolved mono and dipeptides can be matched to an adjacent section of the database sequence if they are isobars. The addition of this re-scoring step can resolve some common de novo sequencing errors and produce identifications that are more accurate.

The sequence homology approach used by the prior art discussed above is limited in several ways when trying to match de novo sequences containing ambiguous regions to database sequences:

This approach can only consider a small number of specific isobaric equivalences, making it difficult to separate de novo sequencing errors from actual sequence modifications.

It is often impossible to analyze marginal de novo sequences derived from poor quality spectra.

These alignment programs cannot easily find post-translational modifications, nor is it possible to search for particular modifications of interest to the researcher.

Significant manual interpretation of BLAST (Altschul, S. F.; Madden, T. L.; Schäffer, A. A.; Zhang, J.; Zhang, Z.; Miller, W.; Lipman, D. J. Nucleic Acids Res. 1997, 25, 3389-3402) and FASTA (Pearson, W. R.; Lipman, D. J. Proc. Natl. Acad. Sci. USA 1988, 85, 2444-2448) results is often required to group peptide hits into likely protein identifications, rendering these programs difficult to use in high-throughput environments.

### SUMMARY OF THE INVENTION

Accordingly, an object of the present invention is to provide improved methods and systems for the identification of macromolecules, including but not limited to proteins, ribonucleic acids, deoxyribonucleic acids, carbohydrates, and lipids.

Another object of the present invention is to provide methods and systems for high-throughput identification of said macromolecules by matching de novo sequences derived from mass spectrometry data of a portion of said macromolecule to homologous macromolecules.

Yet another object of the present invention is to provide methods and systems for identification of potentially complex mixtures of said macromolecules by aligning multiple de novo sequences from all mass spectra for a given experiment to macromolecule sequences in a sequence database.

Still another object of the present invention is to provide methods and systems for the identification of macromolecules from incomplete de novo sequences that cannot account for an entire portion of said macromolecule.

Another object of the present invention is to provide methods and systems for identification of macromolecules that makes mass-based alignments between a de novo sequence and a sequence in a sequence database.

Yet another object of the present invention is to provide methods and systems for identification of macromolecules that makes mass-based alignments from local alignments that can be broken into sub-classes of alignments, scored separately, and linearly combined to create an optimal score that more accurately separates correct identifications from incorrect ones.

Still a further object of the present invention is to provide methods and systems that aligns two de novo sequences from the same portion of said macromolecule to create more accurate consensus sequences, as well as to identify modifications in completely unknown macromolecules by using other de novo sequences as references.

Another object of the present invention is to provide methods and systems that allows sequences of unknown macromolecules to be built from fragments of de novo sequences, including ambiguous mass regions, and those previously unsequenced macromolecules are used for future macromolecule identification.

Yet another object of the present invention is to provide methods and systems that permits macromolecule sequences in the sequence database to be annotated with site-specific modifications to utilize information in databases of known macromolecule modifications.

A further object of the present invention is to provide methods and systems that can be coupled to de novo sequencing programs that are operated in combination as stand-alone macromolecule identification packages, or are used in conjugation with other database-searching programs for independent verification of macromolecule identifications.

These and other objects of the present invention are achieved in a method for identifying sequences of molecules and sequence modifications from mass spectrometry data. At least one de novo sequence is produced from mass spectrometry data of sequences of molecules. At least one mass-based alignment is calculated between each de novo sequence and sequences in a sequence database. The molecular masses of molecules in the de novo sequence are compared to molecular masses of molecules in each sequence in the sequence database. Mass differences of modification sites between the sequence in the sequence database and the de novo sequence that have been identified by the mass-based alignment are interpreted as modifications identified in a modification catalog. At least one match score for the mass-based alignment is calculated that provides an indication of matching between the sequence in the sequence database and the de novo sequence. Sequences in the sequence database are identified from mass-based alignments in response to the match scores. Identifications of sequences in the sequence database are grouped from at least one de novo sequence into an identified macromolecule list that agrees with the de novo sequencing results.

In another embodiment of the present invention, a method is provided for identifying sequences of molecules and sequence modifications from mass spectrometry data. At least one de novo sequence is produced from mass spectrometry data of sequences of molecules. At least one mass-based alignment is calculated between each de novo sequence and sequences in a sequence database. The molecular masses of molecules in the de novo sequence are compared to molecular masses of molecules in each sequence in the sequence database. Mass differences of modification sites between the sequence in the sequence database and the de novo sequence that have been identified by the mass-based alignment are interpreted as modifications identified in a modification catalog,

In another embodiment of the present invention, a computer readable medium is provided that has stored thereon instructions which, when executed by a processor, cause the processor to, (i) execute a first application that produces at least one de novo sequence from mass spectrometry data of sequences of molecules, (ii) execute a second application that calculates at least one mass-based alignment between each de novo sequence and sequences in a sequence database, wherein the molecular masses of molecules in the de novo sequence are compared to molecular masses of molecules in each sequence in the sequence database, and (iii) execute a third program that interprets mass differences of modification sites between the sequence in the sequence database and the de novo sequence that have been identified by the mass-based alignment as modifications identified in a modification catalog.

In another embodiment of the present invention, a computer based system is provided that implement identification sequences of molecules and sequence modifications from mass spectrometry data. The system includes at least a first processor that executes one or more programs that, (i) produce at least one de novo sequence from mass spectrometry data of sequences of molecules, (ii) execute a second application that calculates at least one mass-based alignment between each de novo sequence and sequences in a sequence database, wherein the molecular masses of molecules in the de novo sequence are compared to molecular masses of molecules in each sequence in the sequence database, and (iii) execute a third program that interprets mass differences of modification sites between the sequence in the sequence database and the de novo sequence that have been identified by the mass-based alignment as modifications identified in a modification catalog.

### BRIEF DESCRIPTION OF THE DRAWINGS

FIG. 1 is a flow chart illustrating one method of the present invention for identifying sequences of molecules and sequence modifications from mass spectrometry data.

FIG. 2 is a schematic diagram illustrating implementation of a computer readable medium of the present invention to implement instructions for the FIG. 1 method.

FIG. 3 is a schematic diagram illustrating a computer based system of the present invention that implement identification sequences of molecules and sequence modifications from mass spectrometry data.

FIG. 4 is a flow chart that illustrates one embodiment of the present invention where for each candidate alignment, amino acids encompassing the short tag match in both the de novo and database sequences are converted into their corresponding mass objects.

FIG. 5 is a flow chart that illustrates another embodiment of the present invention where for each candidate alignment, amino acids encompassing the short tag match in both the de novo and database sequences are converted into their corresponding mass objects.

FIG. 6 illustrates an embodiment of the present where for each local alignment, all possible combinations of the next three masses in each sequence are compared sequentially with a breadth-first search algorithm.

FIG. 7 illustrates an embodiment of the present invention where a de novo sequence generated by Peaks from one MS/MS spectrum aligns to bovine serum albumin with significant homology.

FIG. 8 illustrates an alignment scoring system used by with the methods and systems of the present invention separates correct from incorrect peptide assignments.

FIG. 9 illustrates a breakdown of the identifications made by the methods and systems of the present invention, with SEQUEST, and ProteinLynx/AutoMod.

FIG. 10 illustrates an embodiment of the present invention that aligns to the lactotransferrin protein.

### DETAILED DESCRIPTION OF THE PREFERRED EMBODIMENT

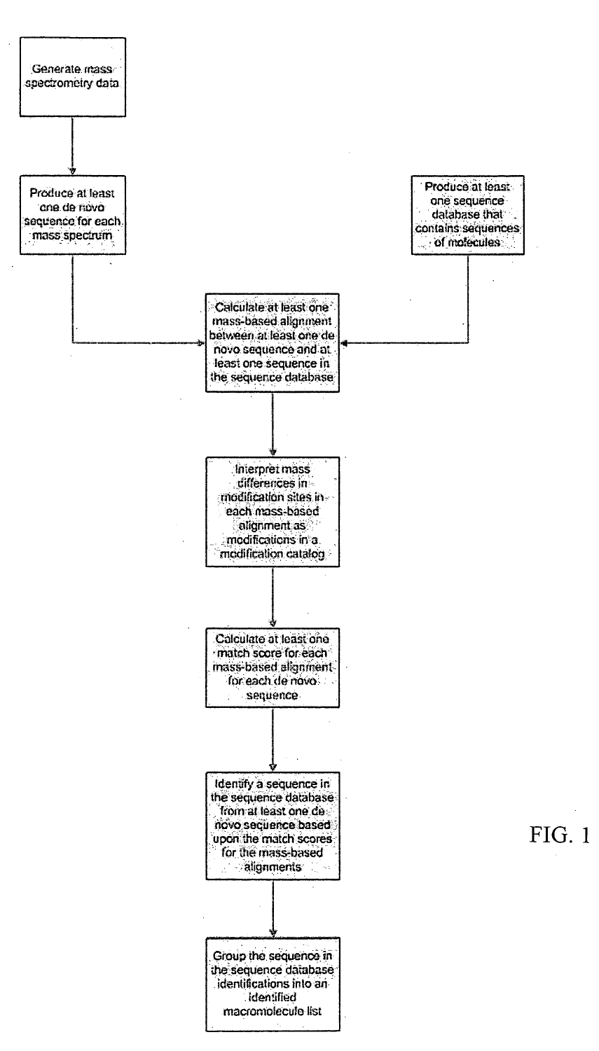

As illustrated in the flowchart of FIG. 1, one embodiment of the present invention provides a method for identifying sequences of molecules and sequence modifications from mass spectrometry data. At least one de novo sequence is produced from mass spectrometry data of sequences of molecules. At least one mass-based alignment is calculated between each de novo sequence and sequences in a sequence database. The molecular masses of molecules in the de novo sequence are compared to molecular masses of molecules in each sequence in the sequence database. Mass differences of modification sites are interpreted between the sequence in the sequence database and the de novo sequence that have been identified by the mass-based alignment as modifications identified in a modification catalog. At least one match score for the mass-based alignment is calculated that provides an indication of matching between the sequence in the sequence database and the de novo sequence. Sequences in the sequence database are identified from mass-based alignments in response to the match scores. Identifications of sequences in the sequence database are grouped from at least one de novo sequence into an identified macromolecule list that agrees with the de novo sequencing results.

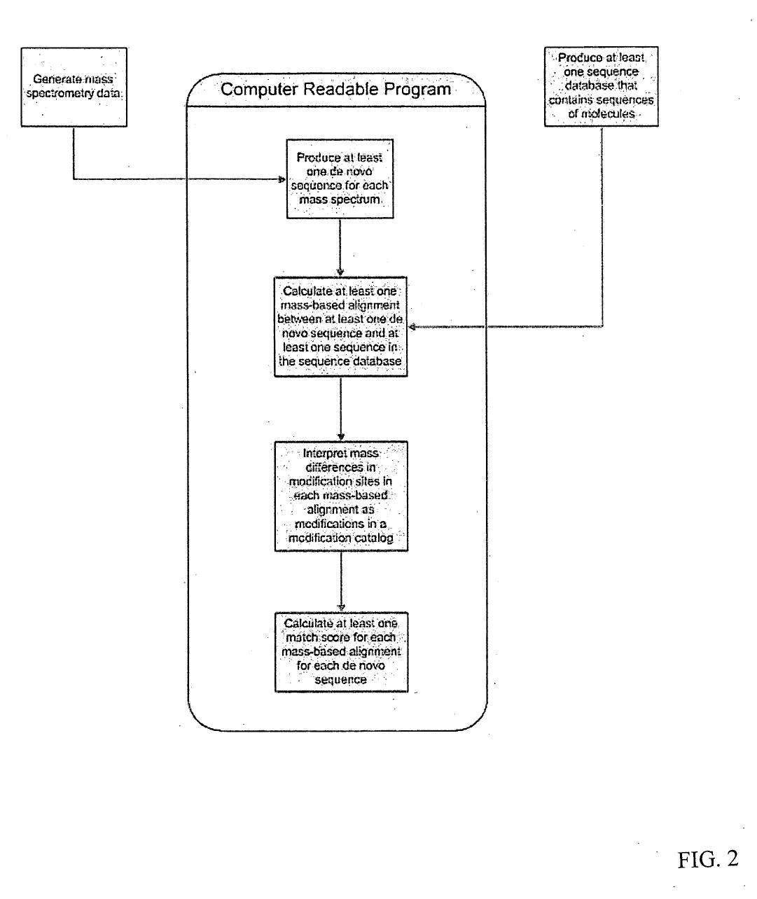

In another embodiment of the present invention, illustrated in FIG. 2, a computer readable medium is provided that has stored thereon instructions which, when executed by a processor, cause the processor to, (i) execute a first application that produces at least one de novo sequence from mass spectrometry data of sequences of molecules, (ii) execute a second application that calculates at least one mass-based alignment between each de novo sequence and sequences in a sequence database, wherein the molecular masses of molecules in the de novo sequence are compared to molecular masses of molecules in each sequence in the sequence database, (iii) execute a third program that interprets mass differences of modification sites between the sequence in the sequence database and the de novo sequence that have been identified by the mass-based alignment as modifications identified in a modification catalog, and (iv) execute a third program that generates at least one match score for the mass-based alignment is calculated that provides an indication of matching between the sequence in the sequence database and the de novo sequence.

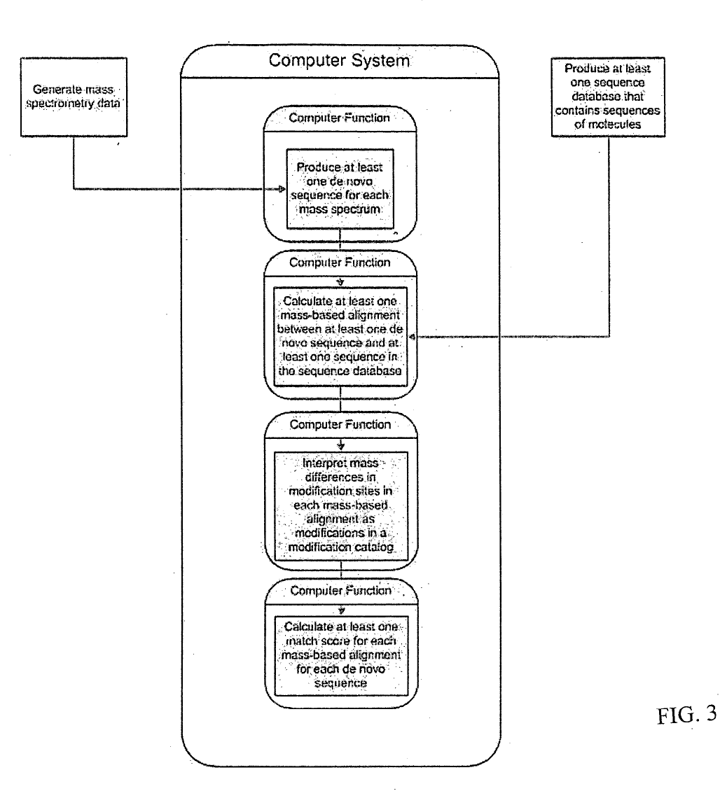

In another embodiment of the present invention, illustrated in FIG. 3, a computer based system is provided that implement identification sequences of molecules and sequence modifications from mass spectrometry data. The system includes at least a first processor that executes one or more programs that, (i) produce at least one de novo sequence from mass spectrometry data of sequences of molecules, (ii) execute a second application that calculates at least one mass-based alignment between each de novo sequence and sequences in a sequence database, wherein the molecular masses of molecules in the de novo sequence are compared to molecular masses of molecules in each sequence in the sequence database, (iii) execute a third program that interprets mass differences of modification sites between the sequence in the sequence database and the de novo sequence that have been identified by the mass-based alignment as modifications identified in a modification catalog, and (iv) execute a third program that generates at least one match score for the mass-based alignment is calculated that provides an indication of matching between the sequence in the sequence database and the de novo sequence.

In one embodiment of the present invention, mass-based alignment of de novo sequences are utilized to accurately identify sequence variations and post-translational protein modifications, thus allowing for these types of searches to succeed in a high-throughput environment. Batch scripting can be used with the methods and systems of the present invention, including the ability to search any number of databases consecutively. XML result files facilitate automatically adding the methods and systems of the present invention alignments into relational databases for the cataloging of protein sequence variations and sites of post-translational modifications. The methods and systems of the present invention can differentiate correct from incorrect hits in a control mixture with a 95% success rate using default parameters, various intermediate score multipliers and score thresholds can be adjusted. This allows for elimination of manual validation.

The methods and systems of the present invention can use the same approach to make every local alignment, and that approach can be broken into sub-classes of alignments, scored separately, and linearly combined to create an optimal score, the methods and systems of the present invention can accurately separate correct identifications from incorrect ones. The methods and systems of the present invention can be used to align two de novo sequences from the same peptide to create more accurate consensus sequences, as well as to identify modifications in unknown proteins by using other de novo sequences as references.

Essentially, this approach allows sequences of unknown proteins to be built from fragments of de novo sequences (including ambiguous mass regions) and those previously unsequenced proteins be used for accurate peptide identification. Furthermore, protein sequences can be annotated with site-specific modifications, which will allow for the future utilization of known protein modifications already being cataloged in databases such as the Human Reference Protein Database (Peri, S.; Navarro, J. D.; Amanchy, R.; Kristiansen, T. Z.; Jonnalagadda, C. K.; Surendranath, V.; Niranjan, V.; Muthusamy, B.; Gandhi, T. K. B.; Gronborg, M.; Ibarrola, N.; Deshpande, N.; Shanker, K.; Shivashankar, H. N.; Prasad, R. B.; Ramya, M. A.; Chandrika, K. N.; Padma, N.; Harsha, H. C.; Yatish, A. J.; Kavitha, M. P.; Menezes, M.; Choudhury, D. R.; Suresh, S.; Ghosh, N.; Saravana, R.; Chandran, S.; Krishna, S.; Joy, M.; Anand, S. K.; Madavan, V.; Joseph, A.; Wong, G. W.; Schiemann, W. P.; Constantinescu, S. N.; Huang, L.; Khosravi-Far, R.; Steen, H.; Tewari, M.; Ghaffari, S.; Blobe, G. C.; Dang, C. V.; Garcia, J. G. N.; Pevsner, J.; Jensen, 0. N.; Roepstorff, P.; Deshpande, K. S.; Chinnaiyan, A. M.; Hamosh, A.; Chakravarti, A.; Pandey, A. Genome Res. 2003, 13, 2363-2371)].

In one embodiment, the methods and systems of the present invention can automatically verify sequencing results against protein sequences in databases. In this approach, the mass-based alignment resources of the present invention help a de novo sequencing program make choices between potential sequence candidates as well as to direct the de novo sequencing program in making more empirically driven decisions. The mass-based alignment of the present invention can be used for a wide number of applications involving the identification of proteins.

In one embodiment, the methods and systems of the present invention are written in Java, run on any platform that can run the Java Runtime Environment (version 1.3). The methods and systems of the present invention have been tested on Windows 2000 and Linux platforms.

In one embodiment, the methods and systems of the present invention align ambiguous MS/MS de novo sequences to protein database sequences. In one embodiment, the methods and systems of the present invention first identify a list of “tags” in a de novo sequence that are all possible combinations of three amino acids not broken by ambiguous mass regions. Tags that are common to both the de novo sequence can be identified, and a given database sequence via a series of string searches where isobaric single amino acids (I/L and K/Q) are replaced with a representative character, similar to the sequence tag method

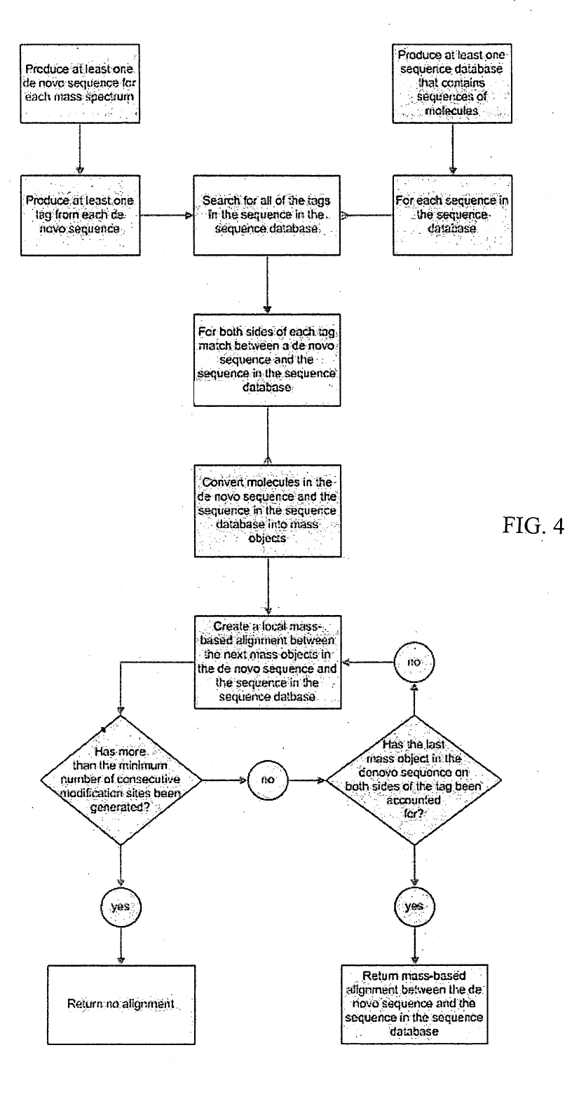

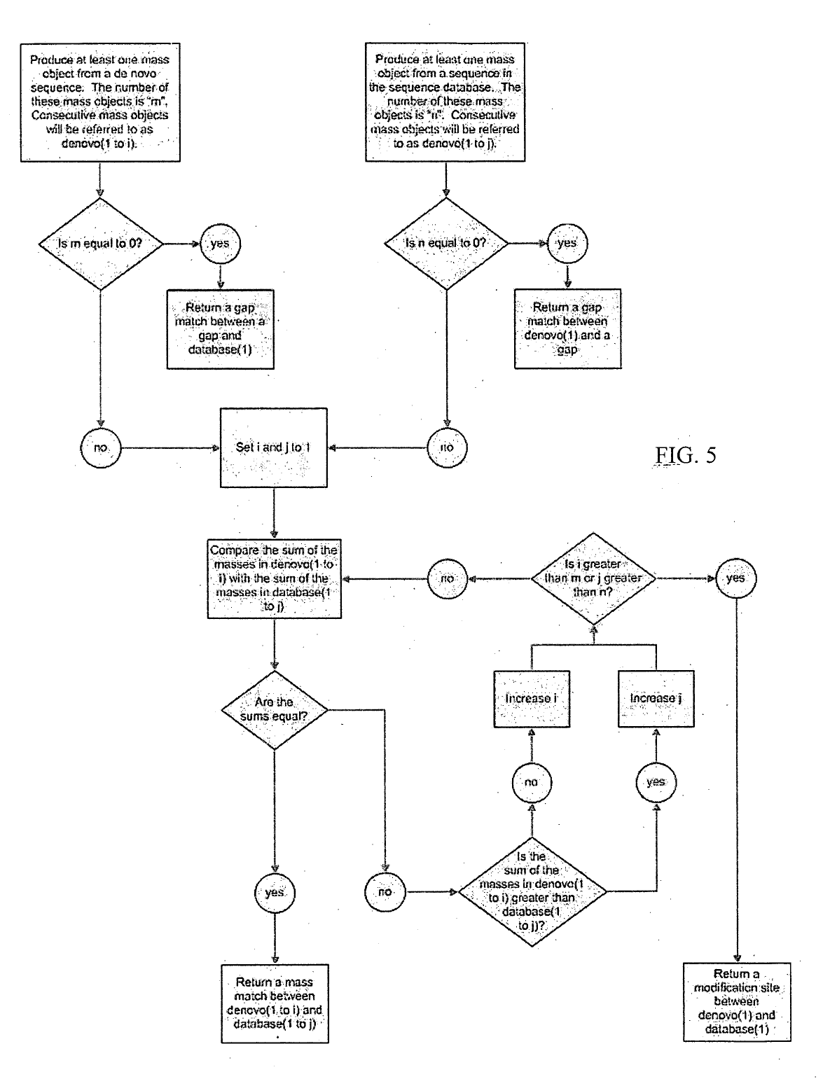

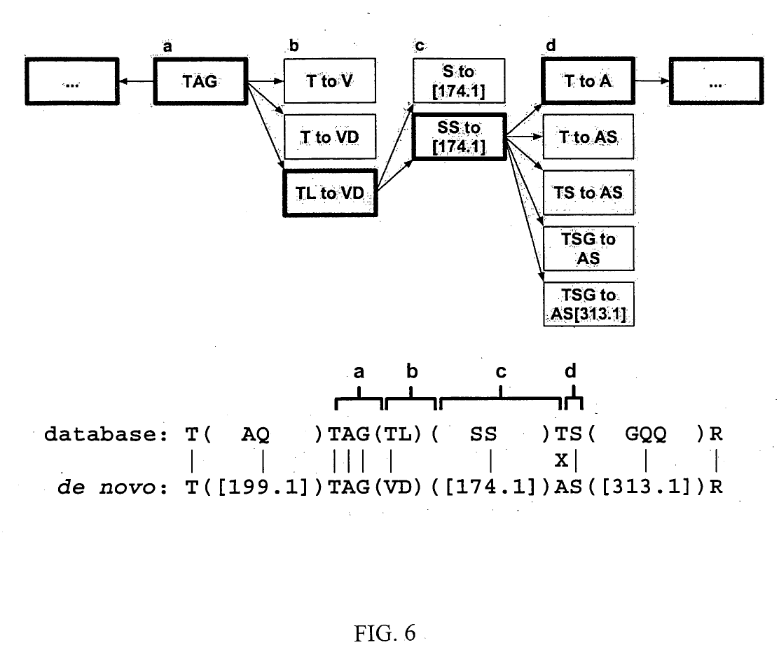

As shown in FIGS. 4 and 5, for each candidate alignment, amino acids encompassing the short tag match in both the de novo and database sequences are converted into their corresponding monoisotopic masses. A series of consecutive local alignments on either side of the tag match are made to form a complete alignment. For each local alignment, all possible combinations of the next three masses in each sequence are compared sequentially with a “breadth-first search” algorithm, as shown in FIG. 6. Initially, the methods and systems of the present invention compare the masses of each of the next residues in the sequences within a fixed mass tolerance. If the masses are unequal, the sequences are compared one “level” deeper, where the mass of one database residue is compared to the mass of two query residues, followed by two database residues versus one query residue, and finally, two database residues versus two query residues. The breadth-first search continues through three levels deep until it finds a mass-match.

By way of illustration, and without limitation, when aligning the isobaric residue combinations of threonine-leucine and valine-aspartic acid, first the mass of Thr (101.0 amu) is compared to the mass of Val (99.1 amu), then Thr (101.0 amu) to sum of Val+Asp (214.1 amu), and finally the sum of Thr+Leu (214.1 amu) to the sum of Val+Asp (214.1 amu), representing a mass-match. The comparison of the mass of Thr+Leu (214.1 amu) to the mass of Val (99.1 amu) does not need to be considered, because it has already been established by the Thr to Val comparison that Thr by itself weighs more than Val.

Masses, or groups of amino acids that were unresolved in the de novo sequence, are treated as if they were single residues that commonly align to two or more residues in the database sequence. If no mass-match can be found by searching through three levels, an amino acid substitution is assumed to have occurred. When a mass-match is made or a substitution is assumed, the breadth-first search is stopped and a new local alignment is initiated starting from the next amino acid in each sequence. The methods and systems of the present invention continue making local alignments until the entire de novo sequence is accounted for. However, only one consecutive substitution is allowed, and the alignment process is terminated if more consecutive substitutions are required to make a match.

The methods and systems of the present invention can be configured to search for residue-specific variable modifications by assigning both the modified and unmodified masses to that residue. Variable N— and C-peptide termini modifications are accounted for in a similar way. Special database amino acid characters, such as B (either asparginine or aspartic acid), Z (either glutamine or glutamic acid), and X (any amino acid) are also implemented: for instance, by assigning the mass of both asparginine and aspartic acid to B. Unknown post-translational protein modifications can be deduced from the shifted masses of specific amino acids, as well as the N— and C-peptide termini.

This approach can find short, isobaric equivalences of an arbitrary residue length, in this case, three consecutive residues or masses, within a given mass tolerance. Although the program execution time grows when more levels are searched, some algorithmic and heuristic-based optimizations have been used to reduce the search time. On average, it takes 9 seconds to search one de novo sequence against the 127873 protein sequences contained in the SwissProt database (Balroch, A.; Boechmann, B. Nucleic Acids Res. 1991, 19, 2247-2249) (release 41.11) on a single Intel Pentium 4 2.0 GHz processor.

In various embodiments of the present invention, alignments and resulting protein identifications are scored. Each local alignment is scored separately and the scores are summed to create a score for the overall peptide alignment. If a mass-match is made in a local alignment, the local alignment score is the average value of the Blosum-90 substitution matrix (Henikoff, S.; Henikoff, J. G. Proc. Natl. Acad. Sci. 1992, 89, 10915-10919)] identities for the database residues in that local alignment. By way of illustration, and without limitation, if an amino acid substitution is made, the local alignment score is the matrix substitution score (S) between the database residue (i) and the de novo sequence residue (j):

$$mass match = \frac{{\sum }_{\begin{matrix}i = database \\ residues\end{matrix}}^{n} {S}_{ii}}{n} substitution = {S}_{ij} \qquad ( 1 )$$

If i contains a residue-specific variable modification, then Sᵢᵢ for that residue is the average identity value (AIV) for the matrix. Similarly, if j is a mass, then Sᵢⱼ for that mass is the average non-identity value (ANV). Gapped-matches, which are only allowed at the beginning and end of the database sequence, are scored as substitutions.

In one embodiment, local alignment mass-matches are broken into three categories: one-to-one, one-to-many or many-to-one, and many-to-many matches, which refer to the number of amino acids in the database and de novo sequences, respectively. In one embodiment, local alignment substitutions are also broken into two categories: common substitutions (with score matrix scores >0) and uncommon substitutions (with score<=0). The peptide alignment score is a linear combination of the summed local alignment scores from these groups:
alignment score =α(Σ_{matches}^{1-to-1})+β(Σ_{matches}^{1-to-m})+χ(_{matches}^{m-to-m})+δ(Σ_{substitutions}^{common})−ε(Σ_{substitutions}^{uncommon})−φ(Σ_{matches}^{gapped}) (2)
where α has been assigned to 1.2, β to 1. 1, χ to 0.9, δ to 1.0, ε to 5.0, and φ to 5.0. These values were empirically derived by analyzing MS/MS spectra derived from human amniotic fluid proteins. In the future, these weights can be statistically tuned for greater resolving power. For reference, the first four terms are always positive, while the last two terms are always negative.

As with CIDentify, information about the enzymatic digestion is used to modify alignment scores. With trypsin, for example, the alignment score is augmented by 3.0*AIV for each terminus of the candidate peptide that matches a tryptic cleavage site (at lysine or arginine). If the candidate peptide indicates a non-tryptic cleavage, the alignment score is decreased by 1.5*ANV for each unmatched terminus. Similarly, the score is decreased by ANV for each lysine or arginine present inside the matched database sequence, representing missed cleavage sites. Other enzymes can be considered in a similar fashion.

Peptide matches with alignment scores over 85 are accepted as correct identifications. Example peptide matches with their corresponding alignments and alignment scores can be found in a supplementary file on the web (Additional results and analysis can be found in the supplementary file on the web at http://medir.ohsu.edu/˜geneview/publication/opensea/). Peptides with long sequences typically have larger scores, however, due to the requirements placed on the actual generation of the alignments, long sequences are generally more difficult to match, justifying their higher score. We've found that factoring the peptide length into the scoring function does not significantly improve the separation of correct from incorrect matches.

The methods and systems of the present invention can include an automatic results compiler that assists in protein identification. The results compiler is similar to ProteinProphet (Nesvizhskii, A. I.; Keller, A.; Kolker, E.; Aebersold, R. Anal. Chem. 2003, 75, 4646-4658), another algorithm developed for database-searching programs that detects proteins using “Occam's Razor” to combine complex peptide identifications into protein hits. The Occam's Razor approach assumes that the simplest combination of proteins that explains the spectral data is the correct interpretation. In order to find the simplest explanation, the methods and systems of the present invention can first identify a list of spectra that can be uniquely assigned to a single protein. By way of illustration, and without limitation, this is done by ranking each peptide with an alignment score above 85 by a “delta score”, which is the difference between the scores of the first and second best alignments for that spectrum. The spectrum with the largest delta score is assigned to the protein corresponding to its best alignment. Two alignments for the same de novo sequence with a score difference of less than 20 are considered to match equally well.

Therefore, all other spectra that match to the protein in question with a delta score of less than 20 are assigned to that protein. Of the remaining spectra, the spectrum with the next largest delta score is then considered and assigned to the protein it matches best. This process is repeated through all of the uncontested identifications. In this manner peptides that match multiple proteins equally well are assigned to the protein with the strongest single peptide evidence (greatest delta score). Two proteins that match the same peptides with the same scores are considered “degenerate” and are grouped together.

In one embodiment, the methods and systems of the present invention score each protein as the sum of the scores of the alignments that match independent regions of that protein. De novo sequences from MS/MS spectra that match the same region of a protein but have different precursor masses (often representing modified peptides) or have different charges are also considered independent. Otherwise, if two de novo sequences align to the same region of a single protein, only 10% of the alignment score for the second sequence is added to the protein score, as these additional identifications often do not provide any new evidence for the protein.

Once the proteins have been identified from the spectra, the remaining unmatched de novo sequences are then realigned to only the identified proteins. In one specific embodiment, the remaining unmatched de novo sequences have alignment scores below 85. The alignments are made using different parameters tuned specifically to find peptides that were poorly sequenced. By way of illustration, and without limitation, five mass levels are searched to identify isobaric equivalent regions for each local alignment, while the length of tags required to initiate an alignment is decreased to two. Furthermore, two consecutive substitutions are allowed. Again by way of illustration, and without limitation, re-alignment matches with alignment scores above 85 are accepted and matches with scores between 85 and 60 are flagged for manual interpretation or verification by a cross correlation method (such as SEQUEST). This approach is similar to the retroactive search done by ProteinLynx via the AutoMod subroutine.

### EXAMPLE 1

### Sample Preparation and LC/MS/MS Spectra Acquisition

In this example, three types of samples were used to test the methods and systems of the present invention. The known protein control mixture was obtained by combining ten purified proteins of varying molecular weight and physiochemical properties. Bos taurus insulin, ubiquitin, cytochrome c, superoxide dismutase, beta-lactoglobulin A, serum albumin, and immunoglobulin G, as well as Equus caballus myoglobin, Armoracia rusticana peroxidase, and Gallus gallus conalbumin were obtained from Ciphergen (Fremont, Calif.). The proteins were combined with urea, reduced with dithiothreitol, and alkylated with iodoacetamide. The mixture was then digested overnight at 37° C with 1 μg modified trypsin (Promega) per 50 μg protein. The resulting peptide mixture was dissolved in 5% formic acid to 2 pmol of total protein per μL of solution. Twelve 1 pmol samples, twenty-two 2 pmol samples, and a single 4 pmol sample were analyzed with MS/MS.

Homo sapiens and Macaca mulatta amniotic fluid samples containing unknown, sequence-modified proteins were obtained from the Oregon Health & Sciences University with Institutional Review Board approval. Proteins were separated by one-dimensional gel electrophoresis and were visualized by Coomassie staining. Bands from each sample were excised and in-gel digested with trypsin and the peptides were extracted from the gel matrix, filtered (0.22 μm), evaporated, and dissolved in 5% formic acid. One high molecular weight band from each sample was chosen for MS/MS analysis.

A lens sample from a 55-year-old Homo sapiens containing post-translationally modified proteins was also obtained from the Oregon Lyons Eye Bank with Institutional Review Board approval from the Oregon Health & Sciences University. 10 μg of total protein was reduced, alkylated, and trypsin digested. The resulting peptides were diluted with 5% formic acid and 10 μg of total protein was analyzed by MS/MS.

All MS/MS spectra were acquired with a Micromass Q-TOF-2 (Milford, Mass.) quadrupole/time-of-flight hybrid mass spectrometer with an online capillary LC (Waters, Milford, Mass.). Samples were desalted with an in-line C18 trap cartridge (LC Packings, San Francisco, Calif.) and separated on a 75 μm×15 cm C18 IntegraFrit column (Waters, Milford, Mass.). Peptides were injected into the online mass spectrometer through a nanospray source.

### EXAMPLE 2

### De Novo Sequencing and Database Searching

In this example, all MS/MS spectra acquired were de novo sequenced. Peaks 1.3 (Ma, B.; Zhang, K.; Hendrie, C.; Liang, C.; Li, M.; Doherty-Kirby, A.; Lajoie, G. Rapid Commun. Mass Spectrom. 2003, 17, 2337-234; Ma, B.; Zhang, K.; Liang. C. “An Effective Algorithm for the Peptide De Novo Sequencing from MS/MS Spectrum”, The 14th Symposium on Combinatorial Pattern Matching, March 2003, 266-278) (Bioinformatics Solutions Inc., Waterloo, ON Canada) and Lutefisk1900 1.3.2 (Fernandez-de-Cossio, J.; Gonzalez, J.; Betancourt, L.; Besada, V.; Padron, G.; Shimonishi, Y.; Takao, T. Rapid Commun. Mass Spectrom. 1998, 12, 1867-1878; Current versions of Lutefisk are available for download at http://www.hairyfatguy.com/Lutefisk/) de novo sequencing programs were used to test the performance of the methods and systems of the present invention. Both programs were configured to assume that all cysteines were alkylated and that all peptides were tryptically digested. Unlike Lutefisk, Peaks reports full amino acid sequences without unknown mass regions, but does assign each amino acid in the sequence a confidence score. Sequence regions where amino acids had confidences scores below 50% were replaced by the combined mass of those amino acids. Lutefisk reports as many as five de novo sequences for each spectrum. All of these sequences were used to produce a match. Only the top scoring sequence reported by Peaks was used, as generally all of the top five Peaks sequences could be represented by the 50% consensus sequence.

Two database-searching programs, TurboSEQUEST 2.0 (Thermo Finnigan, San Jose, Calif.) and ProteinLynx 2.0 (Denny, R.; Neeson, K.; Rennie, C.; Richardson, K.; Leicester, S.; Swainston, N.; Worroll, J.; Young, P. “The Use of Search Workflows in Peptide Assignment From MS/MS Data”, Association of Biomolecular Resource Facilities, ABRF '02: Biomolecular Technologies: Tools for Discovery in Proteomics and Genomics, Austin, Tex., Mar. 9-12, 2002) (Waters, Milford, Mass.), and one de novo sequence alignment program, CIDentify 1.0.8 (Current versions of CIDentify are available for download at ftp://ftp.virginia.edu/fasta/CIDentify/), were used to benchmark The methods and systems of the present invention. All samples of the control mixture were searched against the SwissProt database (Balroch, A.; Boechmann, B. Nucleic Acids Res. 1991, 19, 2247-2249) (release 41.11) that was modified to include sequences for the control proteins that were selected from the non-redundant reference protein database (Wu, C. H.; Huang, H.; Arminski, L.; Castro-Alvear, J.; Chen, Y.; Hu, Z., Ledley, R. S.; Lewis, K. C.; Mewes, H.; Orcutt, B. C.; Suzek, B. E.; Tsugita, A; Vinayaka, C. R.; Yeh, L. L.; Zhang, J; Barker, W. C. Nucleic Acids Res. 2002, 30, 35-37) (PIR-NREF, release 1.25). The human and rhesus monkey amniotic fluid samples, as well as the human lens sample, were searched against the SwissProt database selected for human proteins.

SEQUEST and ProteinLynx were configured to identify tryptic peptides and search for variably alkylated cysteines. DTASelect (Tabb, D. L.; McDonald, W. H.; Yates, J. R. III J. Proteome Res. 2002, 1, 21-26) was used to identify protein matches from SEQUEST results. Protein matches were accepted with multiple peptide hits having cross correlation scores (Xcorrs) of greater than 1.8, 2.5, and 3.5 for singly, doubly, and triply charged peptides, respectively. In ProteinLynx, protein hits having multiple positive peptide match scores were accepted, and the AutoMod subroutine of ProteinLynx (Denny, R.; Neeson, K.; Rennie, C.; Richardson, K.; Leicester, S.; Swainston, N.; Worroll, J.; Young, P. “The Use of Search Workflows in Peptide Assignment From MS/MS Data”, Association of Biomolecular Resource Facilities, ABRF '02: Biomolecular Technologies: Tools for Discovery in Proteomics and Genomics, Austin, Tex., Mar. 9-12, 2002) was used on all samples to find modified peptides belonging to the identified proteins.

CIDentify assumed fixed alkylations and results with E-values less than 10⁻⁴ were accepted. A version of CIDentifyRC (Johnson, R.; Taylor, J. In Methods in Molecular Biology: Mass Spectrometry of Proteins and Peptides; Chapman, J., Ed.; Humana Press: Totawa, N.J., 2000; Vol. 146, pp 41-62) that was modified to process over 100 de novo sequences at a time was used to identify successfully matched proteins. The methods and systems of the present invention were configured to search for the variable alkylation of cysteines, and protein hits with multiple peptide matches having alignment scores of greater than 85.0 were accepted. Both CIDentify and the methods and systems of the present invention were configured to preferentially identify tryptic peptides. In all searches, matches to keratins and trypsin were ignored as contaminants.

### EXAMPLE 3

### Identification of the Control Mixture Proteins

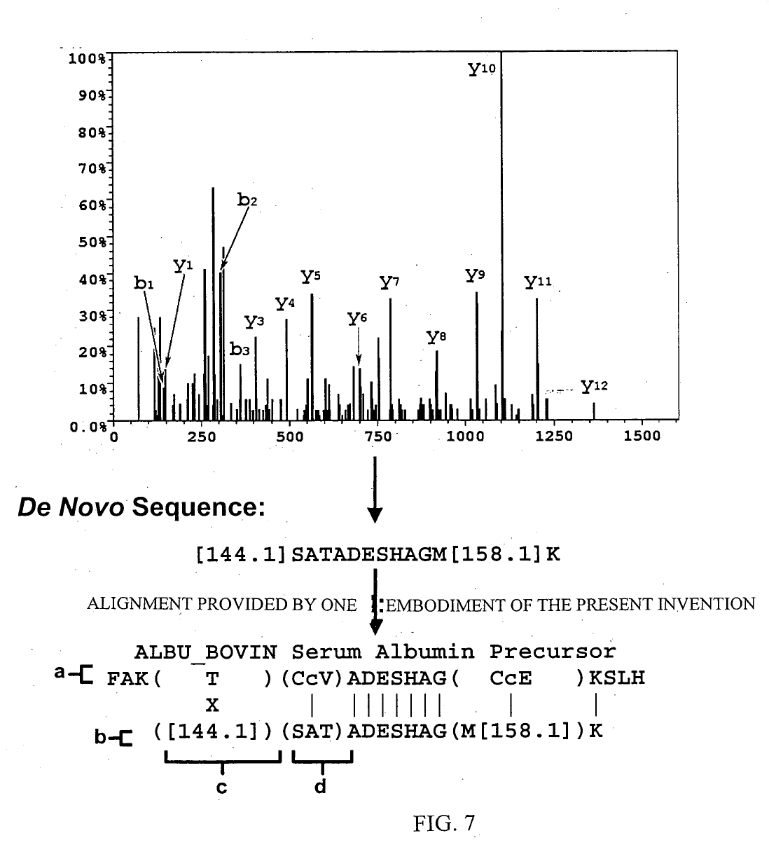

In this example, a mixture of ten tryptically digested proteins was used to evaluate the methods and systems of the present invention. 10685 tandem mass spectra from 35 LC/MS/MS runs of the control mixture were processed with Peaks and then various algorithms of the present invention. As shown in FIG. 7, a de novo sequence generated by Peaks from one MS/MS spectrum is shown to align to bovine serum albumin with significant homology. Peaks accurately identified a three amino acid sequence tag, ADE. From that tag it was established that the methods and systems of the present invention were able to interpret two incorrect regions in the de novo sequence as isobaric equivalents of regions in the protein database sequence, as indicated in parentheses. Variations found by the methods and systems of the present invention represent localized mass discrepancies, which imply the presence of unanticipated modifications or substitutions. In this case, a variation from threonine in the database sequence (101.0 amu) to an unresolved section of the de novo sequence (144.1 amu) was identified. The mass shift of 43.0 amu suggested that the peptide was carbamylated at the N-terminus. This peptide was one of eight from a single LC/MS/MS run that were found to contain this mass shift, which was most likely the result of using urea as a protein denaturant (Stark, G. R.; Stein, W. H.; Moore, S. J. Biol. Chem. 1960, 235, 3177-3181).

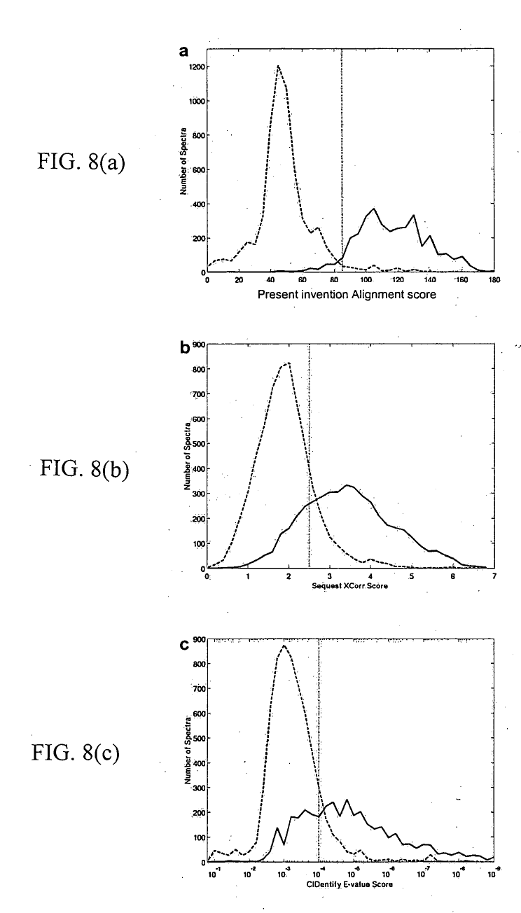

One major requirement for high-throughput MS/MS analysis is an accurate peptide scoring system that can reliably distinguish between correct and incorrect peptide assignments. The accuracy of the default alignment scoring system was estimated by searching de novo sequences generated from all 35 LC/MS/MS runs of the control mixture against the SwissProt protein database (release 41.11), which contained 127863 proteins from various species. Peptide assignments to the ten control proteins were considered unlikely to have occurred by chance, and were therefore assumed to be correct. Conversely, assignments to any other protein were considered incorrect. In one embodiment, illustrated in FIG. 8(a), the alignment scoring system used by with the methods and systems of the present invention separates correct from incorrect peptide assignments.

In one specific embodiment of the methods and systems of the present invention, the default alignment score cutoff of 85 identified 94% of the correct assignments (sensitivity) and eliminated 97% of the incorrect assignments (specificity). For comparison, the sensitivity of the Xcorr score used by SEQUEST was 77%, while the specificity was 85% using minimum Xcorr values of 1.8, 2.5, and 3.5 for peptides of +1, +2, and +3 charge, respectively (FIG. 8b). Similarly, the sensitivity of the CIDentify E-value score was 70% and the specificity was 89% with a minimum score cutoff of 104 (FIG. 8c). Statistical analysis of the methods and systems of the present invention alignment score distributions can be found in the supplementary file on the web (Additional results and analysis can be found in the supplementary file on the web at http://medir.ohsu.edu/˜geneview/publication/opensea/).

A second requirement for high-throughput MS/MS analysis is accurate and easy to interpret protein identifications from peptide matches. The Occam's Razor approach used by the methods and systems of the present invention to identifying protein candidates from the most unambiguous spectral evidence has many benefits. One of which is that a single spectrum is assumed to match only one protein. In the case where the spectrum matches multiple proteins equally, it is assigned to the protein with the greatest evidence for existing in the sample. This is critical to high-throughput analysis because it removes degenerate peptide hits in the case of homologous proteins, which often confound results in large studies. Another benefit is that protein evidence is generated based on how exclusively a single MS/MS spectra can be assigned to that protein based on the delta score, and not on the overall score for that protein. For example, if a single spectrum can be assigned with high confidence to a protein with low overall coverage, the low coverage protein will be reported. This allows low abundance proteins with poor coverage to be found, even if proteins with higher coverage dwarf them. Alternatively, if homologous proteins are expected, the methods and systems of the present invention can be configured to report degenerate peptide matches in proteins with amino acid sequence similarity.

### EXAMPLE 4

### Comparison of the Methods and Systems of the Present Invention to Additional MS/MS Protein Identification Software

One LC/MS/MS run of a 2 pmol control mixture sample was examined in detail to benchmark the number of spectra accurately identified by the methods and systems of the present invention compared to common database-searching programs. Protein identifications of 328 spectra were made by two commonly used database-searching programs, SEQUEST and ProteinLynx, and by two de novo sequence alignment programs, the methods and systems of the present invention and CIDentify. Peaks and Lutefisk were used to provide de novo sequences for both the methods and systems of the present invention and CIDentify. The number of visually verified spectra matching each control protein was tabulated for all of the programs (or combination of programs), and shown in Table 1.

TABLE 1
THE NUMBER OF MS/MS SPECTRA IDENTIFIED AS CONTROL MIXTURE PROTEINS

| | Present | Present | | | | |
|---|---|---|---|---|---|---|
| | invention/ | invention/ | CIDentify/ | CIDentify/ | | ProteinLynx/ |
| Protein Name^{a} | Peaks^{b} | Lutefisk^{c} | Peaks^{d} | Lutefisk^{e} | SEQUEST^{f} | AutoMod^{g} |
| Bovine Serum Albumin | 48 | 14 | 26 | 11 | 40 | 29 |
| Chicken Conalbumin | 27 | 8 | 22 | 4 | 29 | 17 |
| Bovine Immunoglobulin G | 13 | 0 | 7 | 2 | 11 | 14 |
| Equine Myoglobin | 9 | 3 | 4 | 2 | 6 | 8 |
| Bovine B-Lactoglobulin | 8 | 2 | 6 | 2 | 9 | 4 |
| Bovine Superoxide Dismutase | 5 | 2 | 5 | 2 | 9 | 4 |
| Bovine Cytochrome C | 5 | 0 | 5 | 0 | 4 | 2 |
| Bovine Ubiquitin | 4 | 0 | 2 | 0 | 3 | 4 |
| Horseradish Peroxidase | 3 | 0 | 2 | 0 | 6 | 2 |
| Bovine Insulin | 0 | 0 | 0 | 0 | 0 | 0 |
| Total: | 122 | 29 | 79 | 23 | 117 | 84 |

Sequences derived by ProteinLynx automated de novo sequencing (Langridge, J. I.; Millar, A.; Young, P.; O'Malley, R.; Swainston, N.; Skilling, J.; Hoyes, J.; Richardson, K. “A Fully Automated Hierarchical Software Strategy for De Novo Sequencing of Whole Q-T of Electrospray LC-MS/MS Datasets”, Proceedings of the 50th ASMS Conference on Mass Spectrometry and Allied Topics, Orlando, Fla., Jun. 2-6, 2002) were also tested, but both the methods and systems of the present invention and CIDentify generally produced fewer identifications with these sequences than with sequences generated by either Peaks or Lutefisk (data not shown). The methods and systems of the present invention and CIDentify were the only analysis methods that found one of the two tryptic peptides from bovine insulin that were within the mass range of the experiment (not shown in table). However, the match would be difficult to verify because only one peptide from insulin was found.

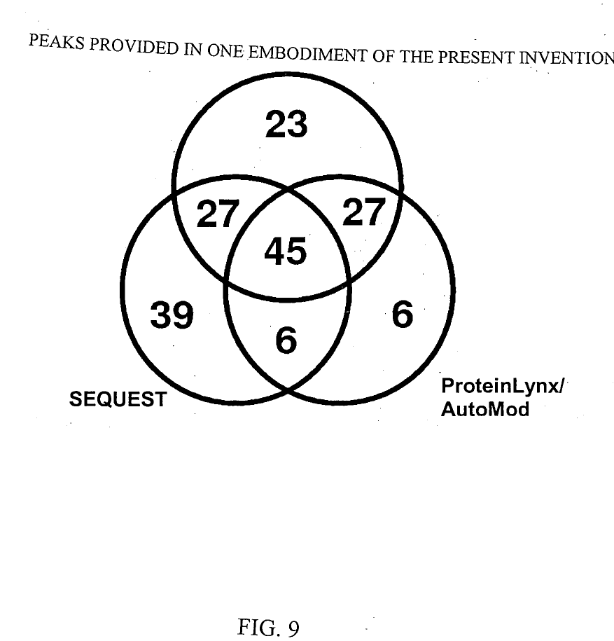

In this example, the methods and systems of the present invention, using de novo sequences derived by Peaks, identified 4% more MS/MS spectra than SEQUEST and 45% more MS/MS spectra than the ProteinLynx search engine using the AutoMod subroutine. A breakdown of the identifications made by the methods and systems of the present invention, SEQUEST, and ProteinLynx/AutoMod is shown in FIG. 9. The methods and systems of the present invention, like CIDentify, identified a comparably low number of MS/MS spectra when using Lutefisk derived de novo sequences. Although both programs identified significantly more peptides when using Peaks de novo sequences versus Lutefisk sequences, the methods and systems of the present invention identified 54% more MS/MS spectra than CIDentify. Only three matches of the identifications made by CIDentify were not found by the methods and systems of the present invention.

In comparison to CIDentify, the increased performance of the methods and systems of the present invention in spectra identification can be the result of many factors. First, the methods and systems of the present invention do not limit the length of its local alignments to single or pairs of residues, and the further interpretation, often results in higher alignment scores for correct matches. Secondly, all alignments of the present invention have stringent, empirically developed criteria requiring that the entire de novo sequence be accounted for, allow for only one consecutive sequence modification, and require that each alignment contain at least one accurately matching sequence tag. Third, the methods and systems of the present invention scoring function separates correct from incorrect matches more reliably than CIDentify, which allows the methods and systems of the present invention to accurately identify lower scoring peptides without introducing a significant number of false positives. The methods and systems of the present invention, and CIDentify, have very distinct approaches to sequence alignment: CIDentify assumes that de novo sequences are generally correct and tries to match them against protein sequences in databases, while presuming that sequence variations are often real. The methods and systems of the present invention, on the other hand, assume that de novo sequences must be verified, and uses protein databases to correct as much of the sequence variation as possible. The methods and systems of the present invention make a more complete and robust interpretations of the actual de novo sequences.

### EXAMPLE 5

### Identification of Unknown, Homologous Proteins

The methods and systems of the present invention can be used to identify proteins that have not been completely sequenced, provided that proteins with close sequence homology are present in the searched databases. Human amniotic fluid was used to represent a mixture of unknown proteins. The amniotic fluid contains fetal proteins that are known to have amino acid variances with their adult homologs. For example, the gamma chain of fetal hemoglobin contains 39 sites of amino acid sequence variation from the adult beta chain (Lorkin, P. A. J. Med. Genet. 1973, 10, 50-64).

A LC/MS/MS run of Homo sapien amniotic fluid proteins from a high molecular weight 1D gel band, generating 416 MS/MS spectra, was analyzed. The spectra were sequenced using Peaks and the resulting sequences were aligned and identified with the methods and systems of the present invention by searching against human proteins in the SwissProt database (9436 proteins). The same spectra were also processed with CIDentify, SEQUEST and ProteinLynx/AutoMod. Protein identifications for each spectrum were manually validated and reported in Table 2(A).

TABLE 2(A)
THE NUMBER OF MS/MS SPECTRA FROM HUMAN (A) AND RHESUS MONKEY (B)
AMNIOTIC FLUID SAMPLES THAT WERE ASSIGNED TO ADULT HUMAN PROTEINS
A

| | Present | | | Protein- | Confirmed/Unconfirmed |
|---|---|---|---|---|---|
| | invention/ | CIDentify/ | | Lynx/ | Amino Acid Variants Found |
| Protein Name | Peaks^{a} | Peaks^{b} | SEQUEST^{c} | AutoMod^{d} | by Present invention/Peaks^{e} |
| Lactotransferrin | 22 | 13 | 5 | 18 | 12/1 |
| Glia Derived Nexin | 11 | 5 | 5 | 10 | 1/1 |
| Serotransferrin | 6 | 4 | 2 | 3 | 2/0 |
| Serum Albumin | 4 | 0 | 5 | 6 | 0/1 |
| Alpha-1-Acid Glycoprotein | 3 | 2 | 2 | 0 | 1/0 |
| Moesin | 3 | 0 | 2 | 0 | 0/0 |
| Myeloperoxidase | 3 | 0 | 2 | 0 | 0/0 |
| Histidine-Rich Glycoprotein | 2 | 0 | 0 | 0 | 1/0 |
| Alpha-1 Antichymotrypsin | 2 | 0 | 2 | 3 | 0/0 |
| Alpha-1 Antitrypsin | 2 | 0 | 2 | 2 | 1/0 |
| Total: | 58 | 24 | 27 | 42 | 18/3 |

Sequence variations identified by the methods and systems of the present invention were confirmed in 18 of the 21 cases by modifying the human protein database to include those sequence variations, and searching the MS/MS spectra against the new database with SEQUEST. For example, the methods and systems of the present invention were used to identify 12 sites of single amino acid variance in amniotic fluid lactotransferrin relative to the human SwissProt sequence (accession number P02788) obtained from non-amniotic fluid samples. ProteinLynx's AutoMod subroutine is an effective modification and sequence variance identification tool and found many of the sequence variant peptides in lactotransferrin that the methods and systems of the present invention reported. However, AutoMod cannot find proteins that have not been identified in the initial database search. The methods and systems of the present invention had a significantly higher peptide and protein identification rate than ProteinLynx/AutoMod. As with the control sample, CIDentify found a subset of the peptides identified by the methods and systems of the present invention, along with two original peptide matches. SEQUEST, as expected, could only find a few unmodified peptides from these proteins, see Table 2(a).

To further this argument, a corresponding LC/MS/MS run, containing 411 MS/MS spectra of Macaca mulatta amniotic fluid proteins, was analyzed in a similar fashion as shown in Table 2(B).

TABLE 2(B)

| | Present | | | Protein- | Confirmed/Unconfirmed |
|---|---|---|---|---|---|
| | invention/ | CIDentify | | Lynx/ | Amino Acid Variants Found |
| Protein Name | Peaks^{a} | Peaks^{b} | SEQUEST^{c} | AutoMod^{d} | by Present invention/Peaks^{e} |
| Lactotransferrin | 25 | 13 | 5 | 16 | 12/1 |
| Glia Derived Nexin | 8 | 5 | 5 | 3 | 1/1 |
| Collagen Alpha 2(I) Chain | 8 | 3 | 0 | 0 | 17/2 |
| Alpha-1 Antitrypsin | 4 | 2 | 2 | 2 | 2/2 |
| Serum Albumin | 4 | 0 | 3 | 2 | 0/0 |
| Gelsolin | 3 | 0 | 0 | 2 | 0/0 |
| 92 kDa type IV Collagenase | 2 | 0 | 2 | 0 | 0/0 |
| Alpha-1 Antichymotrypsin | 0 | 2 | 0 | 0 | 0/0 |
| Total: | 54 | 25 | 17 | 25 | 32/6 |

Although very few rhesus monkey proteins have known sequences, the few known proteins have high sequence homology to their human counterparts. As with the human amniotic fluid sample, sequence variant amino acid sites identified by the methods and systems of the present invention were confirmed with SEQUEST. The methods and systems of the present invention routinely identified peptides with sequence variation from their human analogs and again out performed CIDentify, SEQUEST, and ProteinLynx/AutoMod at peptide and protein identification. For example, only the methods and systems of the present invention and CIDentify could identify collagen alpha 2(I) chain protein, as seven of the eight peptides identified by the methods and systems of the present invention had at least one single amino acid variation.

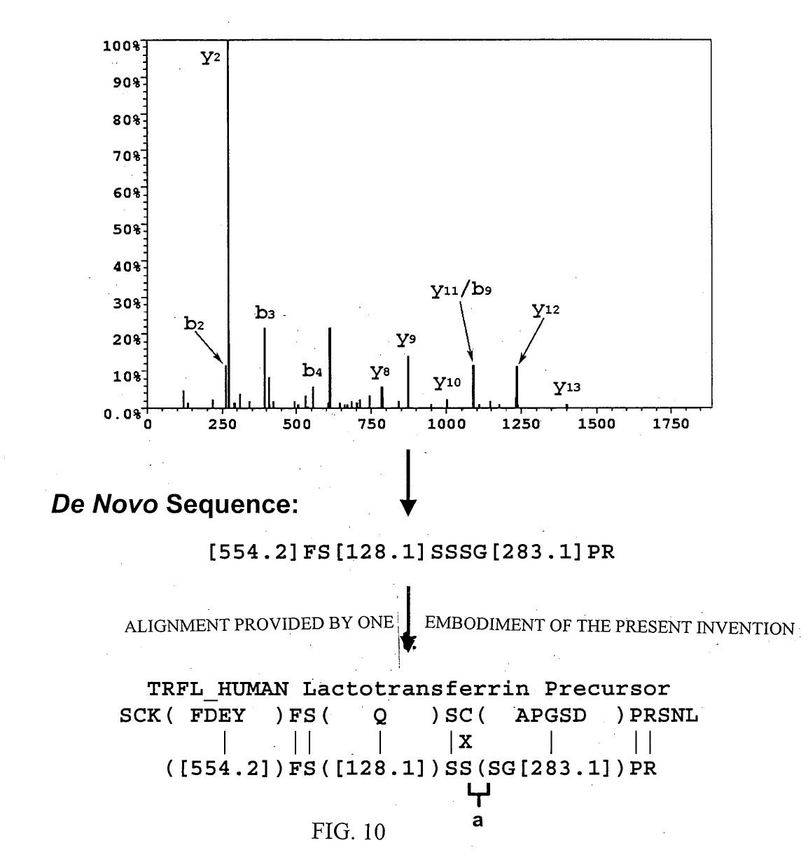

Many other sequence search engines (Shevchenko, A.; Sunyaev, S.; Loboda, A.; Shevchenko, A.; Bork, P.; Ens, W.; Standing, K. G. Anal. Chem. 2001, 73, 1917-1926; Taylor, J. A.; Johnson, R. S. Rapid Commun. Mass Spectrom. 1997, 11, 1067-1075) can identify sequence variations between de novo sequenced peptides and their corresponding sequences in protein databases. One major difficulty is identifying actual sequence variation in the presence of de novo sequencing errors. Because the methods and systems of the present invention's mass-based search algorithm can identify isobaric equivalences of an arbitrary length, it can account for many of the common errors found in sequences generated by Peaks. For example, a poor-quality MS/MS spectrum of a human amniotic fluid peptide was de novo sequenced, and while the resulting sequence contained many ambiguous regions, the methods and systems of the present invention could align it to the lactotransferrin protein, see FIG. 10. The methods and systems of the present invention were able to assign every ambiguous amino acid region to the database sequence, regardless of length. With the unknown regions of the sequence accounted for, a single amino acid variation can be observed at residue 513 in the SwissProt lactotransferrin precursor sequence. The human SwissProt database was modified to reflect this variation and the spectrum was searched against this database with SEQUEST, which confirmed the match (z=2, Xcorr=3.6, dCn=0.37). Additionally, the methods and systems of the present invention assigned the single large peak at 272.2 m/z to a proline-arginine fragment representing a bond cleavage between aspartic acid and proline, which is expected to have enhanced cleavage over other residue pairs in the peptide (Breci, L. A.; Tabb, D. L.; Yates, J. R. III; Wysocki, V. H. Anal. Chem. 2003, 75, 1963-1971). This enhanced cleavage helped support the peptide identification from an otherwise poor-quality spectrum.

### EXAMPLE 6

### Identification of Post-Translational Protein Modifications

Another method using the methods and systems of the present invention is to identify unanticipated in vivo and in vitro protein modifications involves an iterative process where mass differences between the de novo sequence and the database that are associated with particular protein modifications are fed back into the methods and systems of the present invention. The previously unmatched de novo sequences are then searched with the methods and systems of the present invention against the entire database to identify any other peptides that have the same modifications. This two-step process mines information from poor-quality de novo sequences or peptides with multiple modifications that could not otherwise be identified by mass shift alone.

A human lens sample from a 55 year-old male, containing proteins with known post-translational modifications, was used to illustrate this method. Approximately 95% of the protein in the human lens is comprised of just twelve crystallins that do not turnover (MacCoss, M. J.; McDonald, W. H.; Saraf, A.; Sadygov, R; Clark, J. M.; Tasto, J. J.; Gould, K. L.; Wolters, D.; Washburn, M.; Weiss, A.; Clark, J. I.; Yates, J. R. III Proc. Natl. Acad. Sci. USA 2002, 99, 7900-7905). These crystallins undergo post-translational modifications over time,.and because of their long life spans, many tryptic peptides can accumulate two or more modifications per peptide. The methods and systems of the present invention were used to search of the 305 LC/MS/MS spectra generated from this sample generated 85 matches, while identifying 16 peptides with mass variations consistent with either carbamylation, methylation of cysteine, acetylation, oxidation of methionine, or the loss of ammonia or water from a carboxylic acid containing amino acid. Once these identifications were confirmed, the methods and systems of the present invention were configured to specifically find other peptides with these modifications, and six new modification sites were found from 12 new MS/MS matches. All together, the methods and systems of the present invention found six different types of modifications, which are listed in Table 3, and many of the actual modification sites confirm previous reports. For comparison, the AutoMod feature of ProteinLynx identified three types of modifications.

TABLE 3
MODIFICATIONS IDENTIFIED IN THE HUMAN LENS CRYSTALLIN

| | | | | |
|---|---|---|---|---|
| | | Present | | |
| | | invention/ | ProteinLynx/ | |
| | | Peaks | AutoMod | |
| | Nominal | Identified | Identified | |
| Modification^{a} | Mass Shift^{b} | Sites^{c} | Sites^{d} | Example Present invention Alignment^{e} |

| | | | | |
|---|---|---|---|---|
| N-Terminal | 43 | 12 | 7 | NYR( L )VVFELENFQGRRAE |
| Carbamylation | | | | X \|\|\|\|\|\|\|\|\|\|\| |
| | | | | ([156.1])VVFELENFQGR |
| Methylation of Cysteine | 14 | 4 | 0 | GRR( YD |
| | | | | )(Cc)D(Cc)DCADFHTYLSRCNS |
| | | | | \| \| \| \| XX\|\|\|\|\|\|\|\|\| |
| | | | | ([278.1]) (Cc)D(Cc)TMADFHTYLSR |
| N-Terminal Acetylation | 42 | 2 | 2 | MDIAIHH(PW )IRRPF |
| | | | | X:\|\|\|\|\| \| \|\| |
| | | | | SSNLALHH(APD)LR |
| Formation of | −17/−18 | 2 | 0 | VKVQDDFVEIHGKHNE |
| Pyroglutamic acid | | | | :X\|\|\|\|\|\|\|\| |
| | | | | EPDFVELHGK |
| Formation of Succinimide | −17 | 1 | 1 | NYRLVVFELENF( Q )GRRAE |
| | | | | \|\|\|\|\|\|\|X\| \| \|\| |
| | | | | LVVFELEPF([128.1] )GR |
| N-Terminal Acetylation | 42 and 16 | 1 | 0 | MD( V )TI( Q )HP( W |
| and Oxidation of | | | | )FKRTL |
| Methionine | | | | X \|\| \| \|\| \| |
| | | | | \|\| |
| | | | | ([403.2])TL([128.1])HP([186.1])FK |

Cysteines at residues 24 and 26 in gamma crystallin S (Lapko, V. N.; Smith, D. L.; Smith, J. B. Biochem. 2002, 41, 14645-14651), as well as cysteine 82 in beta crystallin A3 (Lapko, V. N.; Smith, D. L.; Smith, J. B. “S-Methylation and glutathionylation of human lens beta crystallins”, Proceedings of the 51st ASMS Conference on Mass Spectrometry and Allied Topics, Montreal, Canada, Jun. 8-12, 2003), were confirmed as methylated in some peptides. Cysteine 185 in beta crystallin A3 was also methylated and SEQUEST verified this previously unidentified methylation site (z=2, Xcorr 3.6, dCn=0.58). Similarly, N-terminal acetylation of alpha crystallin A and beta crystallin B2 were confirmed (Lampi, K. J.; Ma, Z.; Shih, M.; Shearer, T. R.; Smith, J. B.; Smith, D. L.; David, L. L. J. Biol. Chem. 1997, 272, 2268-2275) and the first methionine in alpha crystallin A was variably oxidized (Lampi, K. J.; Ma, Z.; Shih, M.; Shearer, T. R.; Smith, J. B.; Smith, D. L.; David, L. L. J. Biol. Chem. 1997, 272, 2268-2275). An asparagine in beta crystallin B1 had an apparent loss of ammonia to form succinimide, a likely intermediate in non-enzymatic deamidation (Wright, H. T. CRC Crit. Rev. Biochem. 1991, 26, 1-52). An N-terminal glutamine in a peptide from alpha crystallin A was identified as having lost ammonia and an N-terminal glutamic acid in a peptide from alpha crystallin B had similarly lost water. These residues have likely undergone cyclization with the amino terminus during digestion to form pyroglutamic acid (Khandke, K. M.; Fairwell, T.; Chait, B. T.; Manjula, B. N. Int. J. Peptide Protein Res. 1989, 34, 118-123).

All of the modifications were identified without any prior knowledge of the post-translational modifications that are commonly found in lens proteins. In one embodiment, the methods and systems of the present invention can be utilized to automate this search method to mine protein samples for unanticipated post-translational modifications.

The foregoing description of a preferred embodiment of the invention has been presented for purposes of illustration and description. It is not intended to be exhaustive or to limit the invention to the precise forms disclosed. Obviously, many modifications and variations will be apparent to practitioners skilled in this art. It is intended that the scope of the invention be defined by the following claims and their equivalents.

1. A method for identifying sequences of molecules and sequence modifications from mass spectrometry data comprising:
a. producing at least one de novo sequence from mass spectrometry data of sequences of molecules,
b. calculating at least one mass-based alignment between each de novo sequence and sequences in a sequence database, wherein the molecular masses of molecules in the de novo sequence are compared to molecular masses of molecules in each sequence in the sequence database,
c. interpreting mass differences of modification sites between the sequence in the sequence database and the de novo sequence that have been identified by the mass-based alignment as modifications identified in a modification catalog,
d. calculating at least one match score for the mass-based alignment that provides an indication of matching between the sequence in the sequence database and the de novo sequence,
e. identifying sequences in the sequence database from mass-based alignments in response to the match scores, and
f. grouping identifications of sequences in the sequence database from at least one de novo sequence into an identified macromolecule list that agrees with the de novo sequencing results.

2. The method of claim 1, wherein the mass spectrometry data is generated from a tandem mass spectrometer device.

3. The method of claim 1, wherein at least one de novo sequence is an estimated sequence of molecules generated from the mass spectrometry data derived from a sequence of molecules.

4. The method of claim 3, wherein a de novo sequence is a complete or partial sequence of molecules.

5. The method of claim 3, wherein a de novo sequence contains incorrect or unidentifiable region of molecules where the exact sequence of molecules cannot be determined.

6. The method of claim 5, wherein a mass region is the molecular mass of the molecules in an unidentifiable region of molecules.

7. The method of claim 1, wherein at least one molecule is an amino acid and at least one sequence of molecules is a peptide.

8. The method of claim 7, wherein the peptides are derived by an enzymatic digestion of proteins.

9. The method of claim 7, wherein the sequence database is a database of amino acid sequences of proteins.

10. The method of claim 7, wherein the sequence database is a database of amino acid sequences derived from nucleotide sequences.

11. The method of claim 7, wherein the sequence database is a database of de novo peptide sequences.

12. The method of claim 7, wherein the sequence in the sequence database is a particular amino acid sequence in the sequence database.

13. The method of claim 6, further comprising:
a. identifying a sequence in the sequence database with a tag match, and
b. generating a mass-based alignment between a de novo sequence and the sequence in the sequence database.

14. The method of claim 13, wherein a mass-based alignment is a series of consecutive local mass-based alignments on either side of a tag match.

15. The method of claim 14, wherein a tag match is when a tag in the de novo sequence has been shown to be equivalent to a tag in a sequence in the sequence database by way of a tag search.

16. The method of claim 15, wherein a tag search is used to identify a subset of sequences in the sequence database from which to compute mass-based alignments.

17. The method of claim 16, wherein a tag is a sequence of consecutive molecules of a specified length, and the specified length is 2 to 4 molecules in length.

18. The method of claim 16, wherein single molecules of the tag and sequences in the sequence database that have the same nominal weight are represented by a single molecule.

19. The method of claim 14, wherein molecules at either side of the tag match in both the de novo sequence and the sequence of the sequence database are converted into mass objects.

20. The method of claim 19, wherein a mass object is at least one molecular mass and a name for that mass.

21. The method of claim 18, wherein for single molecules, mass objects are assigned the molecular mass of the single molecule.

22. The method of claim 18, wherein for unidentifiable mass regions, mass objects are assigned the molecular mass of the unidentifiable mass region.

23. The method of claim 18, wherein for reference amino acids, which represent multiple amino acids, mass objects are assigned the molecular mass of each amino acid.

24. The method of claim 19, wherein for variably modified amino acids, mass objects are assigned multiple molecular masses.

25. The method of claim 19, wherein mass regions are treated as single molecules with a single molecular mass.

26. The method of claim 19, wherein a gap is a mass object of zero molecular mass that represents no molecule.

27. The method of claim 19, wherein a local mass-based alignment is a matching of at least one consecutive mass object in the sequence in the sequence database and at least one consecutive mass object in a de novo sequence.

28. The method of claim 27, wherein each local mass-based alignment is generated with a breadth-first search, wherein all possible sequential combinations of mass objects of the next specified number of mass objects are compared.

29. The method of claim 28, wherein the specified number of mass objects used in the breadth first search is the search depth.

30. The method of claim 29, wherein the search depth is 3-5.

31. The method of claim 21, wherein the breadth first search is used identify the local mass-based alignment as either a mass match, a substitution, or a gap match:

32. The method of claim 31, wherein the breadth first search first tries to identify a mass match, as a local mass-based alignment where the sum of the molecular masses of the consecutive mass objects in the sequence in the sequence database and the sum of the molecular masses of the consecutive mass objects in a de novo sequence are equal within a specified mass tolerance.

33. The method of claim 31, wherein if there are no mass objects left on the side of the tag match in the sequence in the sequence database, a gap match is identified as a local mass-based alignment between a gap and at least one consecutive mass object in either the sequence in the sequence database or the de novo sequence.

34. The method of claim 31, wherein if a mass match or a gap cannot be identified, then the breadth first search identifies a modification site as a local mass-based alignment where the sum of the molecular masses of the consecutive mass objects in the sequence in the sequence database and the sum of the molecular masses of the consecutive mass objects in a de novo sequence are not equal within a specified mass tolerance.

35. The method of claim 31, wherein the number of mass objects in the de novo sequence and the number of mass objects in the sequence database is minimized.

36. The method of claim 31, wherein the specified mass tolerance is designated by a mass tolerance of a tandem mass spectrometer device that generates the mass spectrometry data.

37. The method of claim 28, wherein a new local mass-based alignment is generated starting from the next molecule in the de novo sequence and the next molecule in the sequence in the sequence database after the last molecule that is matched in the breadth-first search in each respective sequence.

38. The method of claim 37, wherein a series of local mass-based alignments are made until the entire de novo sequence has been accounted for by the sequence in the sequence database in the mass-based alignments.

39. The method of claim 38, wherein a maximum number of consecutive modification sites are performed.

40. The method of claim 39, wherein the maximum number of consecutive modification sites is 1-or 2 local mass-based alignments in length.

41. The method of claim 39, wherein the modification information about modifications is cataloged in a modification catalog.

42. The method of claim 41, wherein the modification information includes at least one of, molecular mass of the modification, a specific molecules where the modification occurs, a frequency of occurrence of the modification at those molecules, wherein the frequency of occurrence is the estimated frequency in nature or a frequency as a sample preparation artifact, a mass object for the modification, which represents the additional mass of the modification to the de novo sequence at those molecules, and the name of the modification, and a modification score for the modification.

43. The method of claim 42, wherein a modification is selected from, an in vivo or in vitro protein, a peptide modification, and an amino acid substitution.

44. The method of claim 43, further comprising: ranking the modifications, wherein the ranking is based on their frequency of occurrence.

45. The method of claim 44, further comprising: identifying a most probable modification in the modification site from the modification catalog by matching elements to elements in modifications in the modification catalog that are selected from at least one of, the mass difference, the molecules in the sequence database in the modification site, and the ranking of the modification in the modifications catalog.

46. The method of claim 45, wherein the mass difference is the difference between the sum of the molecular masses of the consecutive mass objects in the sequence in the sequence database and the sum of the molecular masses of the consecutive mass objects in a de novo sequence in a local mass-based alignment.

47. The method of claim 45, wherein the mass object of an identified modification is inserted into the in the mass-based alignment, which creates a mass match between the de novo sequence and the sequence in the sequence database.

48. The method of claim 38, further comprising: computing a match score of the mass-based alignment, the match score being a measure of how well the sequence in the sequence database matches the de novo sequence.

49. The method of claim 48, wherein a match score is generated from the linear combination of local alignment scores from the series of local mass-based alignments.

50. The method of claim 49, wherein each of a series of consecutive local mass-based alignments receives a score and is classified.

51. The method of claim 50, wherein each local alignment score is generated using a substitution matrix, depending on whether the local alignment is a mass match, a modification site, or a gap match.

52. The method of claim 51, wherein the substitution matrix contains substitution matrix score of least one molecule.

53. The method of claim 52, wherein the substitution matrix identity score is a substitution matrix score between a molecule and itself.

54. The method of claim 53, wherein the substitution matrix substitution score is a substitution matrix score between a molecule and a different molecule.

55. The method of claim 54, wherein the substitution matrix score is the log of the odds score of an identity of a molecule or a substitution between two molecules.

56. The method of claim 52, wherein the local alignment score for a mass match is the average value of the substitution matrix identity scores for all of the molecules in the sequence in the sequence database matched in the local alignment.

57. The method of claim 56, wherein if at least one of the molecules has been modified by a modification, the substitution matrix score for each modified molecule is the modification score for that modification.

58. The method of claim 52, wherein if the local mass-based alignment is a match between only one mass object from the sequence in the sequence database, and only one mass object from the de novo sequence, and that those mass objects represent single molecules, then the local alignment score for a substitution is the substitution matrix substitution score between the molecule in the sequence in the sequence database and the molecule in the de novo sequence.

59. The method of claim 52, wherein the local alignment score for a substitution is the number of molecules in the substitution in the sequence in the sequence database multiplied by the average value of the substitution matrix substitution scores.

60. The method of claim 52, wherein the local alignment score for a gap match is the number of molecules in the gap match in the de novo sequence multiplied by the average value of the substitution matrix substitution scores.

61. The method of claim 48, wherein if the termini of the de novo sequence are expected to be specific molecules., the match score is increased if the termini of the mass-based alignment match the expected specific molecules.

62. The method of claim 48, wherein if the termini of the de novo sequence are expected to be specific molecules, the match score is decreased if the termini of the mass-based alignment do not match the expected specific molecules, or if expected specific molecules are present inside the mass-based alignment.

63. The method of claim 1, further comprising utilizing an approach that interprets matches between sequences in the sequence database and de novo sequences, which have been scored by a match score, as an identified macromolecule list and assigns a macromolecule score to each sequence in the identified macromolecule list.

64. The method of claim 63, wherein the match score is a measure of how well the sequence in the sequence database matches the de novo sequence.

65. The method of claim 64, wherein de novo sequences that match at least one sequence in the sequence database are classified as either discriminating de novo sequences or non-discriminating de novo sequences, the de novo sequences are inserted into a de novo sequence list, and the de novo sequences in the de novo sequence list are ranked by their delta scores.

66. The method of claim 65, wherein the delta score is computed for the de novo sequence as the difference between the match scores of the first and second matches to sequences in the sequence database for that de novo sequence. If that de novo sequence only matches one sequence in the sequence database, the delta score is the match score for that match.

67. The method of claim 66, wherein discriminating de novo sequences have a delta score greater than or equal to the delta score threshold and non-discriminating de novo sequences have a delta score less than the delta score threshold.

68. The method of claim 67, wherein the delta score threshold for the de novo sequence is between 0% and 25% of the match score of the highest scoring match between a sequence in the sequence database and that de novo sequence.

69. The method of claim 67, All matches between a sequence in the sequence database and a de novo sequence with match scores less than the match score of the highest scoring match between a sequence in the sequence database and that de novo sequence minus the delta score threshold are discarded.

70. The method of claim 60, wherein the sequence in the sequence database, which matches best to the discriminating de novo sequence in the de novo sequence list with the greatest delta score, is added to the identified macromolecule list. This de novo sequence is then moved from the de novo sequence list to that sequence.

71. The method of claim 70, wherein all non-discriminating de novo sequences in the de novo sequence list that match to that sequence in the identified macromolecule list are moved from the de novo sequence list to that sequence.

72. The method of claim 71, wherein the process of 1 is repeated until all discriminating de novo sequences in the de novo sequence list are removed from the de novo sequence list.

73. The method of claim 72, wherein all sequences in the sequence database that match to non-discriminating de novo sequences still in the de novo sequence list are added to the identified macromolecule list, and the non-discriminating de novo sequences still in the de novo sequence list are moved to those sequences.

74. The method of claim 73, wherein a macromolecule score is generated for every sequence in the identified macromolecule list.

75. The method of claim 74, wherein the macromolecule score is a linear combination of the de novo macromolecule scores of the de novo sequences that have been assigned to that sequence.

76. The method of claim 64, a new sequence database is generated containing only the sequences in the sequence database that are listed in the identified macromolecule list.

77. The method of claim 76, wherein de novo sequences that do not match any sequence in the original sequence database are re-analyzed by calculating a mass-based alignment between each de novo sequence in question and sequences in the new sequence database, as described in claim 1 in a way that the search space explored by the mass-based alignment algorithm is increased.

78. The method of claim 77, further comprising: decreasing the specified length of tags.

79. The method of claim 77, further comprising: increasing the search depth.

80. The method of claim 77, further comprising: increasing the maximum number of consecutive substitutions.

81. The method of claim 64, wherein de novo sequences that do not match any sequence in the original sequence database are re-analyzed by calculating a mass-based alignment between each de novo sequence in question and sequences in a different sequence database, as described in claim 1.

82. A method for identifying sequences of molecules and sequence modifications from mass spectrometry data comprising:
a. producing at least one de novo sequence from mass spectrometry data of sequences of molecules,
b. calculating at least one mass-based alignment between each de novo sequence and sequences in a sequence database, wherein the molecular masses of molecules in the de novo sequence are compared to molecular masses of molecules in each sequence in the sequence database,
c. interpreting mass differences of modification sites between the sequence in the sequence database and the de novo sequence that have been identified by the mass-based alignment as modifications identified in a modification catalog, and
d. calculating at least one match score for the mass-based alignment that provides an indication of matching between the sequence in the sequence database and the de novo sequence.

83. The method of claim 82, further comprising: identifying sequences in the sequence database from mass-based alignments in response to the match scores.

84. The method of claim 83, further comprising: grouping identifications of sequences in the sequence database from at least one de novo sequence into an identified macromolecule list that agrees with the de novo sequencing results.

85. A computer readable medium having stored thereon instructions which, when executed by a processor, cause the processor to perform:
a. executing a first application that produces at least one de novo sequence from mass spectrometry data of sequences of molecules,
b. executing a second application that calculates at least one mass-based alignment between each de novo sequence and sequences in a sequence database, wherein the molecular masses of molecules in the de novo sequence are compared to molecular masses of molecules in each sequence in the sequence database,
c. executes a third program that interprets mass differences of modification sites between the sequence in the sequence database and the de novo sequence that have been identified by the mass-based alignment as modifications identified in a modification catalog, and
d. executes a fourth program that calculates at least one match score for the mass-based alignment that provides an indication of matching between the sequence in the sequence database and the de novo sequence.

86. The computer readable medium of claim 85, wherein the processor further executes a fifth program that identifies sequences in the sequence database from mass-based alignments in response to the match scores.

87. The computer readable medium of claim 86, wherein the processor further executes a sixth program that groups identifications of sequences in the sequence database from at least one de novo sequence into an identified macromolecule list that agrees with the de novo sequencing results.

88. A computer based system that implements identification sequences of molecules and sequence modifications from mass spectrometry data, comprising at least a first processor that executes one or more programs that:
a. produces at least one de novo sequence from mass spectrometry data of sequences of molecules,
b. executing a second application that calculates at least one mass-based alignment between each de novo sequence and sequences in a sequence database, wherein the molecular masses of molecules in the de novo sequence are compared to molecular masses of molecules in each sequence in the sequence database, and
c. executes a third program that interprets mass differences of modification sites between the sequence in the sequence database and the de novo sequence that have been identified by the mass-based alignment as modifications identified in a modification catalog, and
d. executes a fourth program that calculates at least one match score for the mass-based alignment that provides an indication of matching between the sequence in the sequence database and the de novo sequence.

89. The computer based system of claim 88, wherein at least a first processor executes one or more programs that identifies sequences in the sequence database from mass-based alignments in response to the match scores.

90. The computer based system of claim 89, wherein at least a first processor executes one or more programs that groups identifications of sequences in the sequence database from at least one de novo sequence into an identified macromolecule list that agrees with the de novo sequencing results.
